(19) United States
(12) Patent Application Publication
(10) Pub. No.: US 2009/0203975 A1
(43) Pub. Date: Aug. 13, 2009

(54) METHOD AND DEVICE FOR TREATING HEEL PAIN OF A PATIENT

(76) Inventor: Stephen L. Barrett, Anthem, AZ (US)

Correspondence Address: BARNES & THORNBURG LLP, 11 SOUTH MERIDIAN, INDIANAPOLIS, IN, 46204, US

(21) Appl. No.: 12/030,560

(22) Filed: Feb. 13, 2008

(51) Int. Cl. A61B 19/00; A61B 5/00; A61N 1/00

(52) U.S. Cl. 600/306; 600/557; 600/368; 607/46

(57) ABSTRACT

A method and tool for treating heel pain of a patient. The method includes determining a medical procedure to be performed on the patient based on the number of etiologies, the time span, and the severity of the patient's heel pain. The medical procedure performed may include collecting and processing a sample of the patient's blood to retrieve growth factors therefrom, excising degenerative tissue of the patient's plantar fascia, injecting the patient's growth factors into the patient's plantar fascia, and applying an electrical acoustic shockwave treatment to the patient's plantar fascia. The tool includes a trocar and a rasp configured to be received in the trocar. The rasp includes a file defined at a distal end and configured to extend through an opening of the trocar.

### TECHNICAL FIELD

The present disclosure relates generally to a method and device for treating heel pain.

### BACKGROUND

Heel pain may be caused by many different factors ranging from simple mechanical problems within a patient's heel to complex metabolic diseases. Many heel pain occurrences are a result of degeneration in the tissue of a patient's plantar fascia. The plantar fascia is a broad ligamentous band of tissue, which extends from the bottom of the heel to the front portion of the patient's foot distinguish

When the tendonous tissue in a patient's plantar fascia degrades, the patient may begin to experience pain in the corresponding heel. Eventually, this tendonous tissue may degrade to the point where the patient's body can no longer heal itself. Healthcare providers refer to this condition as plantar fasciitis and/or plantar fasiosis.

### SUMMARY

According to one aspect, a method for treating heel pain may include determining the number of etiologies of a patient's heel pain. For example, the number of etiologies may be determined by analyzing the patient's responses to a questionnaire designed to identify multiple etiology heel pain syndrome. The method may also include determining the time span of the heel pain of the patient. To determine the time span of the patient's heel pain, a healthcare provider may assess the duration of the heel pain of the patient and the presence of previous treatment for heel pain received by the patient. The method may further include determining the severity of the heel pain of the patient. For example, the severity of the heel pain of a patient may be determined by performing an ultrasound procedure on a heel of the patient and determining the thickness of hypoechoic tissue in the heel of the patient.

The method may also include performing a medical procedure on the patient wherein the medical procedure is selected based on the previously determined number of etiologies, time span, and severity of the heel pain of the patient. The medical procedure may be a partial plantar fasciectomy in some embodiments. For example, the medical procedure may be performed by collecting a sample of the patient's blood and processing the sample to retrieve the patient's growth factors therefrom. The medical procedure may also include performing an ultrasound procedure on the heel of the patient and identifying degenerative areas of the patient's plantar fascia. The medical procedure may further include excising at least a portion of the degenerative tissue of the patient's plantar fascia with a rasp. Additionally, the medical procedure may include injecting the patient's growth factors into the patient's plantar fascia. Further, the medical procedure may include applying an electrical shockwave treatment to the patient's plantar fascia. For example, the shockwave treatment may include applying about 2,000 extracorporeal electrical shocks to the patient's plantar fascia.

According to another aspect, a method for performing a partial plantar fasiectomy procedure may include collecting a sample of a patient's blood. The method may also include processing the sample to retrieve the patient's growth factors therefrom. The method may further include performing an ultrasound procedure on the patient's heel and identifying degenerative areas of the patient's plantar fascia.

Additionally, the method may include excising the degenerative tissue of the patient's plantar fascia. For example, the degenerative tissue of the patient's plantar fascia may be excised by using a rasp. Excising the degenerative tissue of the patient's plantar fascia may also comprise using a small gauge percutaneous cannulated technique. The method may further include injecting the patient's growth factors into the patient's plantar fascia. For example, injecting the patient's growth factors into the patient's plantar fascia may be performed subsequent to excising the degenerative tissue of the patient's plantar fascia.

Further, the method may include applying an electrical shockwave treatment to the patient's plantar fascia. For example, applying an electrical shockwave treatment to the patient's plantar fascia may comprise applying about 2,000 extracorporeal shocks to the patient's plantar fascia. Additionally, applying an electrical shockwave treatment to the patient's plantar fascia may be performed subsequent to identifying the degenerative area of the patient's plantar fascia by performing an ultrasound procedure, excising the degenerative tissue of the patient's plantar fascia, and injecting the patient's growth factors into the patient's plantar fascia.

According to a further aspect, a surgical tool for use in the performance of a partial planar fasiectomy procedure may include a trocar and a rasp. The trocar may include a base and an elongated shaft extending from the base. The elongated shaft may include an internal passageway defined therein. Additionally, the elongated shaft may include an opening defined toward a distal end. The opening may be in communication with the internal passageway. The rasp may be coupled to the trocar. The rasp may include an elongated shaft and a file defined toward a distal end of the elongated shaft. The elongated shaft may be received in the internal passageway of the trocar. The file may extend through the opening of the elongated shaft. In some embodiments, the trocar may include an inner sidewall that defines an aperture in the base. In such embodiments, the inner sidewall may include an elongated slot defined therein. Additionally, in such embodiments, the rasp may include a rail. The rail of the rasp may be received in the elongated slot of the trocar.

### BRIEF DESCRIPTION OF THE DRAWINGS

The detailed description particularly refers to the following figures, in which:

FIG. 1 is a simplified flowchart of one embodiment of a process for treating heel pain of a patient;

FIG. 2 is one embodiment of a questionnaire and chart for determining the number of etiologies of the heel pain of a patient;

FIG. 3 is one embodiment of a chart for determining the stage of the heel pain of a patient;

FIG. 4 is one embodiment of a chart for determining the severity of the heel pain of a patient;

FIG. 5 is a simplified flowchart of one embodiment of a process for performing a partial plantar fasiectomy procedure;

FIG. 6 is an exploded perspective view of a tool for performing a partial plantar fasiectomy procedure;

FIG. 7 is a perspective view of a trocar of the tool of FIG. 6;

FIG. 8 is a end elevation view of the trocar of FIG. 7;

FIG. 9 is a perspective view of a rasp of the tool of FIG. 6; and

FIG. 10 is a perspective view of the tool of FIG. 6 in an assembled configuration.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

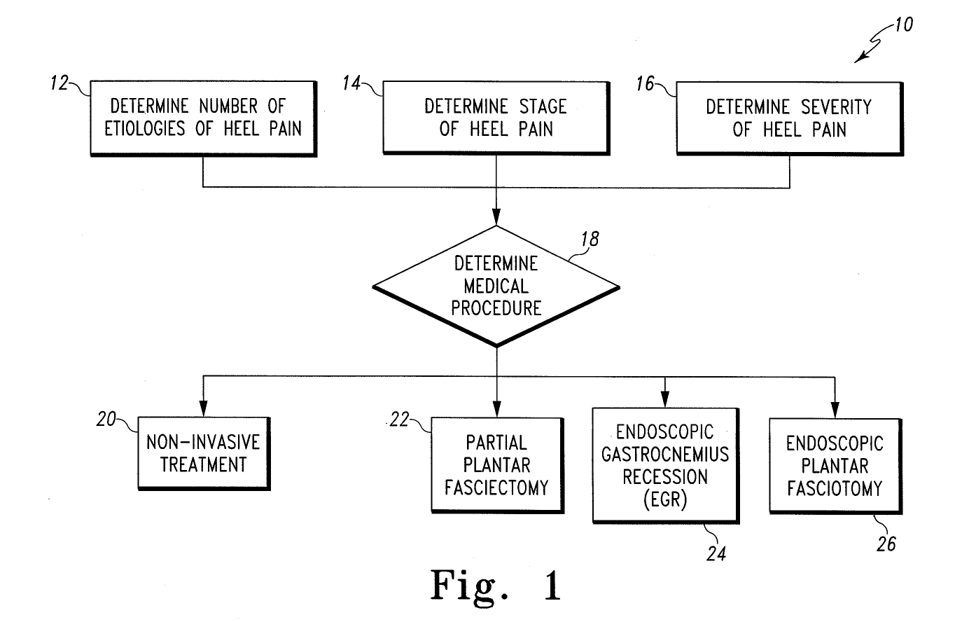

Referring to FIG. 1, an algorithm 10 for treating heel pain of a patient includes a number of heel pain determination steps 12, 14, 16. In process step 12, the number of etiologies of the patient's heel pain is determined. Additionally, the stage of the patient's heel pain is determined in process step 14 and the severity of the patient's heel pain is determined in process step 16. The process steps 12, 14, 16 may be performed in any order. Additionally, any one or more of the process steps 12, 14, 16 may be performed contemporaneously with one or more of the other process steps 12, 14, 16.

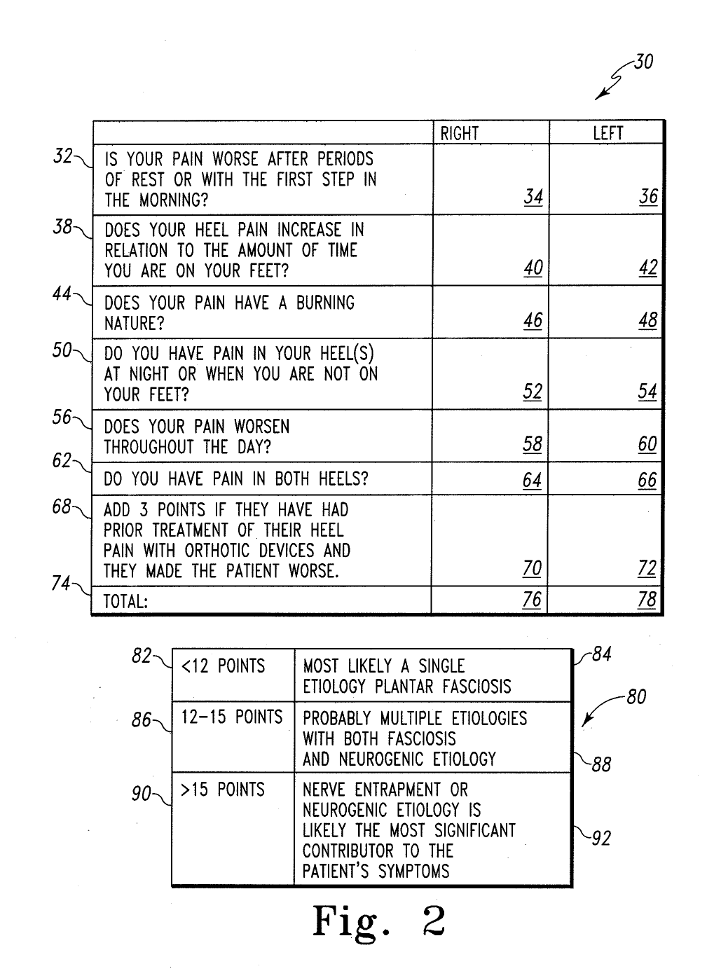

As illustrated in FIG. 2, in one embodiment, the number of etiologies of the patient's heel pain may be determined by a healthcare provider via use of a questionnaire 30. The questionnaire 30 includes a number of questions to identify multiple etiologies of heel pain. The healthcare provider may present each question of the questionnaire 30 to the patient and note the responses provided by the patient thereon. Alternatively, the questionnaire 30 may be provided to the patient to review and fill out.

In some embodiments, the questionnaire 30 includes a question 32 designed to determine if the patient's heel pain is worse after periods of rest. The questionnaire 30 also includes a response area 34 in which the patient's response in regards to the patient's right heel is recorded. Additionally, the questionnaire 30 includes a response area 36 in which to record the patient's response in regards to the patient's left heel. In one particular embodiment, a value of 0 is entered in the response areas 34, 36 to indicate that the patient's heel pain is not worse after periods of rest and a value of 1 is entered in the response areas 34, 36 to indicate that the patient's heel pain is worse after periods of rest. However, in other embodiments, different numerical ranges may be used.

In some embodiments, the questionnaire 30 further includes a question 38 designed to determine if the patient's heel pain increases in relation to the amount of time that the patient is on his or her feet. Again, the questionnaire 30 includes response areas 40, 42 in which to record the patient's responses in regards to the patient's right and left heels respectively. In one particular embodiment, a value of 0 is entered in the response areas 40, 42 to indicate that the patient's pain does not increase in relation to the amount of time the patient is on his or her feet and a value of 1 is entered in the response areas 40, 42 to indicate that the patient's pain does increase in relation to the amount of time the patient is on his or her feet. However, in other embodiments, different numerical ranges may be used.

Additionally, in some embodiments, the questionnaire 30 includes a question 44 designed to determine if the patient's heel pain consists of a burning sensation. Again, the questionnaire 30 includes response areas 46, 48 in which to record the patient's responses in regards to the patient's right and left heels respectively. In one particular embodiment, a value of 0 is entered in the response areas 46, 48 to indicate that the patient's pain does not consist of a burning sensation and a value of 1 is entered in the response areas 46, 48 to indicate that the patient's pain does consist of a burning sensation. However, in other embodiments, different numerical ranges may be used.

Further, in some embodiments, the questionnaire 30 includes a question 50 designed to determine if the patient experiences heel pain during times of rest or general inactivity when the patient is not on his or her feet. Again, the questionnaire 30 includes response areas 52, 54 in which to record the patient's responses in regards to the patient's right and left heels respectively. In one embodiment, a value of 0 is entered in the response areas 52, 54 to indicate that the patient does not experience heel pain during times of rest and a value of 1 is entered in the response areas 52, 54 to indicate that the patient does experience heel pain during times of rest. However, in other embodiments, different numerical ranges may be used.

In some embodiments, the questionnaire 30 further includes a question 56 designed to determine if the patient's heel pain worsens throughout a typical day. Again, the questionnaire 30 includes response areas 58, 60 in which to record the patient's responses in regards to the patient's right and left heels respectively. In one particular embodiment, a value of 0 is entered in the response areas 58, 60 to indicate that the patient's heel pain does not throughout the day and a value of 1 is entered in the response areas 58, 60 to indicate that the patient's heel pain does worsen throughout the day. However, in other embodiments, different numerical ranges may be used.

Further, in some embodiments, the questionnaire 30 includes a question 62 designed to determine if the patient experiences pain in both heels. Again, the questionnaire 30 includes response areas 64, 66 in which to record the patient's responses in regards to the patient's right and left heels respectively. In one embodiment, a value of 0 is entered in the response areas 64, 66 to indicate that the patient's does not experience heel pain in the corresponding heel and a value of 1 is entered in the response areas 64, 66 to indicate that the patient does experience heel pain in the corresponding heel. However, in other embodiments, different numerical ranges may be used.

Additionally, in some embodiments, the questionnaire 30 includes a question 68 designed to determine if the patient has received prior treatment for heel pain, which included the use of an orthotic device, and whether that treatment made the patient's heel pain worse. Again, the questionnaire 30 includes response areas 70, 72 in which to record the patient's responses in regards to the patient's right and left heels respectively. In one particular embodiment, a value of 0 is entered in the response areas 70, 72 to indicate that the patient has not received previous and unsuccessful heel pain treatment with orthotic devices and a value of 3 is entered in the response areas 70, 72 to indicate that the patient has received previous and unsuccessful heel pain treatment with orthotic devices. However, in other embodiments, different numerical ranges may be used.

The questionnaire 30 further includes areas 76, 78 in which to record the total value of the numerical responses in regards to each of the patient's heels. In one embodiment, a total value between 0 and 9 is entered in area 76. The value entered in area 76 may be determined by adding the values entered into response areas 34, 40, 46, 52, 58, 64, 70, which correspond to the patient's responses regarding the patient's right heel. Likewise, in one embodiment, a total value between 0 and 9 is entered in area 78. The value entered in area 78 is determined by adding the values entered into response areas 36, 42, 48, 54, 60, 66, 72, which correspond to the patient's responses regarding the patient's left heel.

Furthermore, to aid the healthcare provider in determining the number of etiologies of the patient's heel pain the healthcare provider may utilize a diagnosis chart 80 as illustrated in FIG. 2. The diagnosis chart 80 may include several ranges of values, which include all of the feasible total values that may be entered in areas 76, 78 of the questionnaire 30 based on adding the numerical results of the patient's responses recorded in the response areas of the questionnaire 30. In one embodiment, the diagnosis chart 80 includes the following three numerical ranges: a less than twelve range 82, a between twelve and fifteen range 86, and a greater than fifteen range 90. The diagnosis chart 80, further includes a description of the etiologies of the patient's heel pain corresponding to each numerical range 82, 86, 90. In one embodiment, the description 84 of a single etiology plantar fasciosis corresponds to numerical range 82, the description 88 of multiple etiologies with both fasciosis and neurogenic etiology corresponds to numerical range 86, and the description 92 of nerve entrapment or neurogenic etiology corresponds to numerical range 90. However, in other embodiments, different ranges and corresponding etiological descriptions may be used.

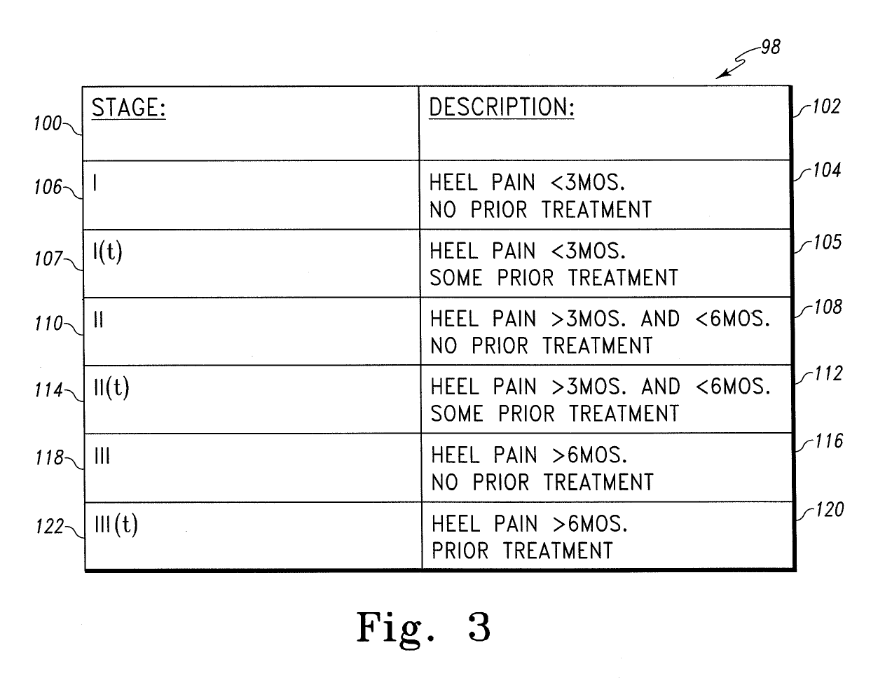

Referring back to FIG. 1, the algorithm 10 for treating heel pain of a patient includes the process step 14 wherein the stage of the patient's heel pain is determined. A healthcare provider may determine the stage of a patient's heel pain by determining the time span of the patient's heel pain. In some embodiments, a healthcare provider may also inquire as to the existence of previous treatment undergone by the patient to alleviate the heel pain to determine the stage of the patient's heel pain. Additionally, a healthcare provider may determine the stage of the patient's heel pain via a stage chart 98, as illustrated in FIG. 3.

In one embodiment, the stage chart 98 includes a column with a cell 100 indicating that the column contains various stages of patient heel pain. For example, the stage chart 98 includes the following six stages: I 106, I(t) 107, II 110, II(t) 114, III 118, and III(t) 122. The stage chart 98 further includes a column with a cell 102 indicating that the column contains various descriptions of patient heel pain. Accordingly, the healthcare provider may determine the time span of the patient's heel pain, locate the corresponding description in the column identified by cell 102, and then determine the corresponding stage in the column identified by cell 100. Additionally, in some embodiments, the healthcare provider may further determine whether the patient received previous treatment for the heel pain to determine the stage.

In one embodiment, the stage chart 98 contains the following six descriptions of time span of and previous treatment for patient heel pain: heel pain for a duration of less than three months with no prior treatment 104, heel pain for a duration of less than three months with some prior treatment 105, heel pain for a duration of between three and six months with no prior treatment 108, heel pain for a duration of between three and six months with prior treatment 112, heel pain for a duration of greater than six months with no prior treatment 116, and heel pain for a duration of greater than six months with prior treatment 120. For example, if a healthcare provider determined that a patient had experienced heel pain for five months and the patient had received previous treatment for the heel pain, the healthcare provider would determine, using the stage chart 98, that the stage of the patient's heel pain is II(t) 114. However, in other embodiments different stage designations and corresponding stage descriptions may be used.

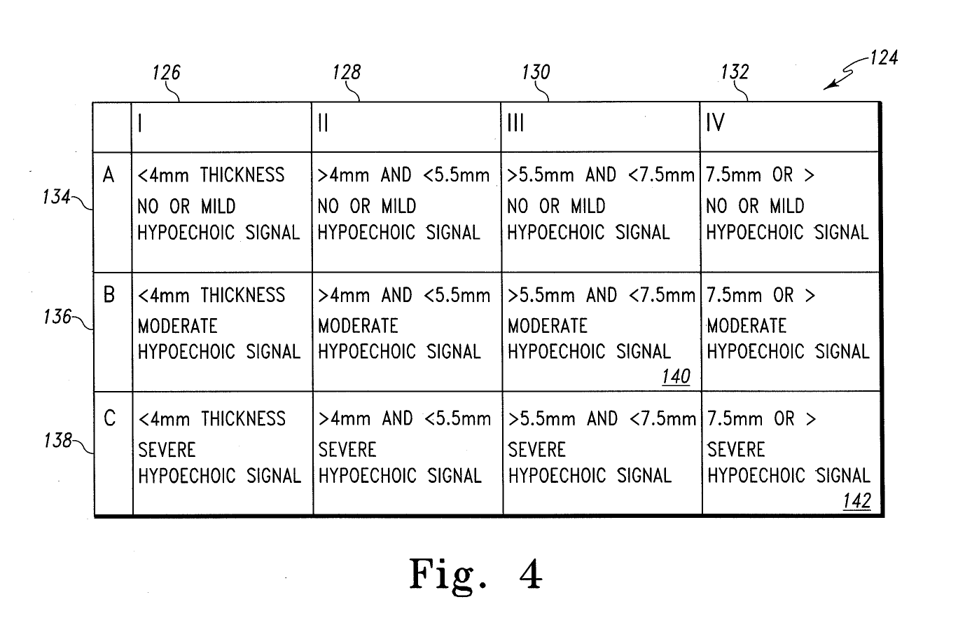

Referring back to FIG. 1, the algorithm 10 for treating heel pain of a patient also includes the process step 16 wherein the severity of the patient's heel pain is determined. In some embodiments, a healthcare provider may determine the severity of a patient's heel pain by performing an ultrasound procedure on the patient's heel. Additionally, a healthcare provider may determine the severity of the patient's heel pain via a severity chart 124, as illustrated in FIG. 4.

In one particular embodiment, the healthcare provider may perform an ultrasound procedure on the patient's heel and determine the thickness of degenerative tissue in the plantar fascia of the patient's heel and the quality of the signal registered during the ultrasound procedure, which corresponds to the degenerative tissue. Once the healthcare provider determines the thickness and signal quality, the severity chart 124 may be used to categorize the severity of the patient's heel pain.

For example, in one embodiment as illustrated in FIG. 4, the severity chart 124 includes four columns, each containing a different range of thicknesses of the degenerative plantar fascia tissue of a patient. The first column, identified by a cell 126 and containing the character I, corresponds to the thickness range of less than four millimeters. The second column, identified by a cell 128 and containing the characters II, corresponds to the thickness range of between four millimeters and five-and-one-half millimeters. The third column, identified by a cell 130 and containing the characters III, corresponds to the thickness range of between five-and-one-half millimeters and seven-and-one-half millimeters. Furthermore, the fourth column, identified by a cell 132 and containing the characters IV, corresponds to the thickness range of seven-and-one-half millimeters or greater. However, in other embodiments, different column identifiers and ranges of thicknesses may be used.

Additionally, in one embodiment, the severity chart 124 includes three rows, each of which contains a different ultrasound signal quality registered for the degenerative tissue of a patient's plantar fascia. The first row, identified by a cell 134 and containing the character A, corresponds to no or a mild hypoechoic signal. The second row, identified by a cell 136 and containing the character B, corresponds to a moderate hypoechoic signal. Furthermore, the third row, identified by a cell 138 and containing the character C, corresponds to a severe hypoechoic signal. However, in other embodiments, different row identifiers and signal quality descriptions may be used.

The signal quality (e.g., mild, moderate, severe, etc.) of the ultrasound may be determined by comparing the ultrasound image to a grayscale chart, which associates signal quality to grayscale brightness areas. For example, in some embodiments, this analysis may be performed by comparing, within the sensitivity range of the transducer head (factoring out all anisotropic signals), the brightest white area within a particular ultrasound scan or image to the darkest area in the same image field and referencing such areas to the grayscale chart. As discussed above, the grayscale chart correlates the level of brightness of such areas to a signal level quality. Additionally, in some embodiments, the hypoechoic areas of the ultrasound image are correlated with the power doppler, which measures (without units) relative movement (e.g., blood flow) in the particular image area.

Thus, a healthcare provider using the severity chart 124 would categorize the severity of a patient's heel pain as III(B) if the healthcare provider performed an ultrasound on the patient and determined that the thickness of degenerative plantar fascia tissue in the patient's heel was six millimeters and that the ultrasound device registered a moderate hypoechoic signal. This result would correspond to a cell 140, located in the severity chart 124, which indicates that the thickness of the degenerative tissue is within the range of about five-and-one-half millimeters and seven-and-one-half millimeters and that the ultrasound registered a moderate hypoechoic signal for the corresponding tissue. Similarly, a healthcare provider using the severity chart 124 would categorize the severity of a patient's heel pain as IV(C) if the healthcare provider determined that the thickness of degenerative plantar fascia tissue in the patient's heel was eight millimeters and that the ultrasound registered a severe hypoechoic signal. This result would correspond to a cell 142, located in the severity chart 124, which indicates that the thickness of the degenerative tissue is greater than about seven-and-one-half millimeters and that the ultrasound registered a severe hypoechoic signal for the corresponding tissue.

Referring back to FIG. 1, the algorithm 10 for treating heel pain of a patient further includes the process step 18 wherein the medical procedure to be performed on the patient in order to alleviate the patient's heel pain is determined. To make this determination, a healthcare provider considers the results of process steps 12, 14, and 16. Based on the results of process steps 12, 14, and 16, the healthcare provider may determine to perform one or more of the process steps 20, 22, 24, or 26.

In process step 20 of the algorithm 10, a non-invasive treatment to alleviate the patient's heel pain may be performed. In one particular embodiment, this non-invasive treatment may include biomechanically stabilizing the foot of the patient with a custom designed orthotic device. In another particular embodiment, this non-invasive treatment may include a growth factor infiltration technique designed to restore the degenerative tissue of the patient's plantar fascia. In other embodiments, other types of non-invasive treatments may be performed such as, for example, physical therapy, iontophoresis, micro vas/electric stimulation, and/or other methods for stabilizing the patient's heel such as strapping the patient's foot to a support device.

In process step 24, an endoscopic gastrocnemius recession (EGR) surgical procedure may be performed on the patient. In one particular embodiment, the endoscopic gastrocnemius recession surgical procedure is a minimally invasive procedure, which results in an amount of correction comparable to traditional invasive or open procedures for the treatment of gastrocnemius equinus.

In process step 26 of the algorithm 10, an endoscopic plantar fasciotomy surgical procedure may be performed on the patient. In one particular embodiment, the endoscopic plantar fasciotomy surgical procedure includes performing a procedure in which the patient's plantar fascia is incised from the patient's heel bone. This may be performed by making an incision into the heel of the patient, inserting a canula into the incision, inserting an endoscope into the canula to allow viewing of the anatomy of the patient's plantar fascia, and incising the patient's plantar fascia from the patient's heel bone via a cutting instrument. In this embodiment, the cutting instrument and canula are then removed and the patient's heel is bandaged. However, in other embodiments, other steps may be taken to release the patient's plantar fascia from the patient's heel bone.

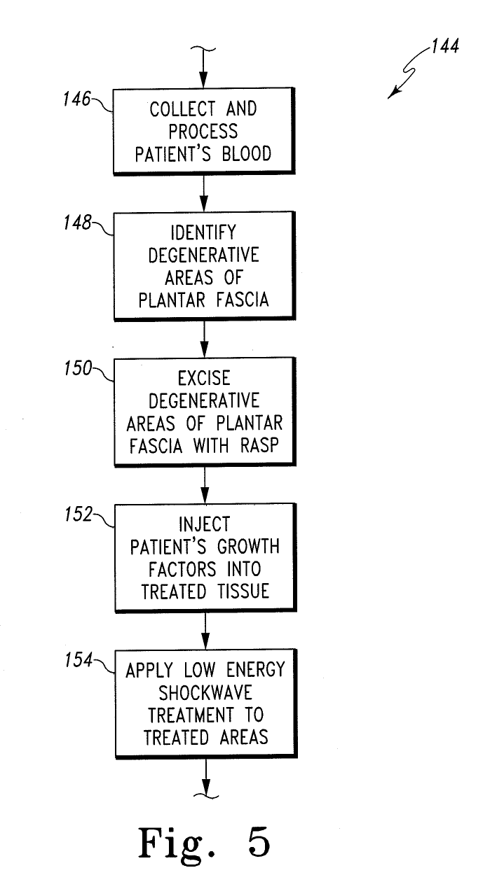

Referring now to process step 22 of the algorithm 10, a partial plantar fasciectomy procedure may be performed on the patient in this process step. To do so, as illustrated in FIG. 5, an algorithm 144 performing a partial plantar fasciectomy procedure may be performed by the healthcare provider. The algorithm 144 for performing a partial plantar fasciectomy procedure includes a process step 146 in which a sample of the patient's blood is collected and processed. In one particular embodiment, the process step 146 includes withdrawing a sample of blood from the patient's arm. This may be done while the patient is asleep via a general anesthetic. Additionally, in one particular embodiment, the process step 146 includes processing the patient's collected blood sample to retrieve autologous platelet concentrate, which contains the patient's growth factors.

Further, the algorithm 144 for performing a partial plantar fasciectomy procedure includes a process step 148 for identifying degenerative areas of the patient's plantar fascia. In one particular embodiment, the degenerative areas of a patient's plantar fascia may be identified by performing an ultrasound procedure on the patient's heel. Additionally, in some embodiments, the ultrasound procedure may be used to determine the degree of degeneration in the patient's plantar fascia tissue. Further, the process step 148 may, in some embodiments, be performed using a high-resolution ultrasound device with power Doppler capabilities.

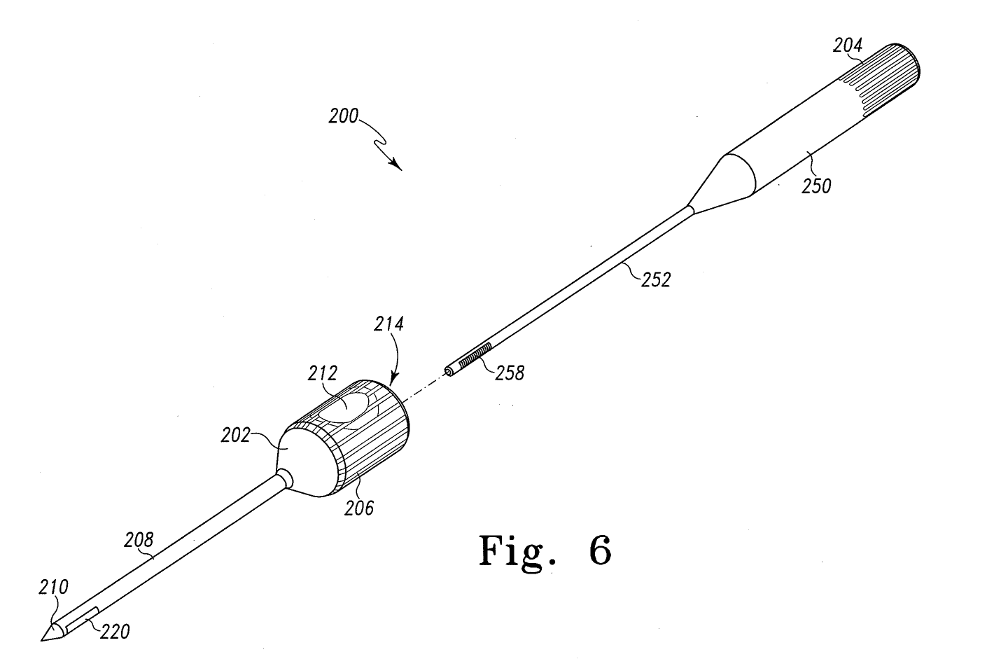

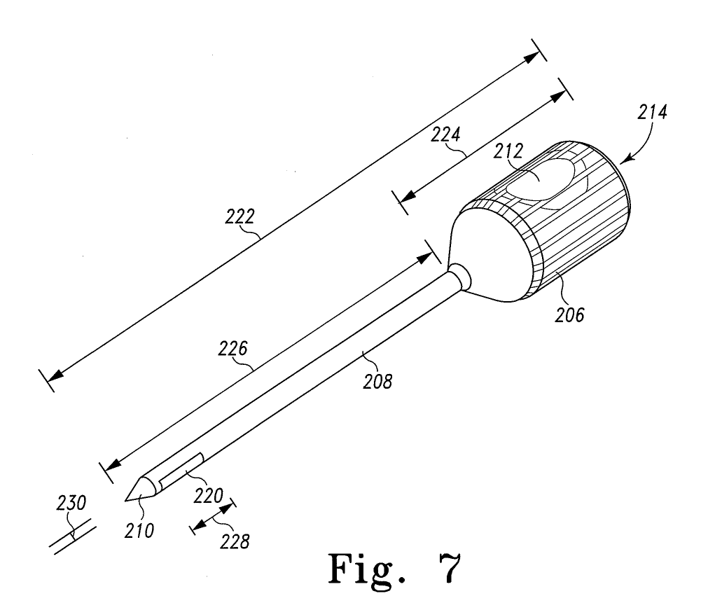

The algorithm 144 also a process step 150 in which the degenerative areas of the patient's plantar fascia is excised. In one particular embodiment, the process step 150 includes excising the degenerative areas of the patient's plantar fascia using a rasp tool such as the rasp tool 200 illustrated in and discussed below in regard to FIGS. 6-10. Moreover, the rasp may be inserted into the patient's degenerative tissue using a small gauge percutaneous cannulated technique. In other embodiments, the process step 150 of excising the degenerative areas of the patient's plantar fascia may be accomplished by visually monitoring the patient's degenerated plantar fascia tissue. For example, a healthcare provider may perform a real-time high-resolution diagnostic ultrasound procedure on the patient's heel while excising the degenerative areas of the patient's plantar fascia. In some embodiments, this ultrasound procedure may include power Doppler visualization.

Further, the algorithm 144 for performing a partial plantar fasciectomy procedure includes a process step 152 wherein the patient's previously collected and processed growth factors are injected into the plantar fascia tissue of the patient. In one particular embodiment, the process step 152 includes injecting the patient's growth factors into the patient's tissue via a canula, which may have been previously inserted into the patient's plantar fascia. Additionally, in some embodiments, a healthcare provider may inject the patient's growth factors into the patient's tissue while visually monitoring this tissue. For example, this may be accomplished by performing a real-time high-resolution diagnostic ultrasound procedure.

Additionally, the algorithm 144 for performing a partial plantar fasciectomy procedure includes a process step 154 in which a shockwave treatment is applied to the treated areas of the patient's heel to stimulate the growth factors previously injected into the patient's heel in process step 152. For example, in one particular embodiment an electrical acoustic shockwave treatment may be used. The energy level and number of shockwave applications may be selected such that the platelets are lysed and the grow factors are stimulated. For example, in some embodiments, process step 152 may include the application of about 2000 extracorporeal, low energy electrical shockwaves. Moreover, in one particular embodiment, a healthcare provider may apply the shockwave treatment to the patient's heel using a Swiss DolorClast low energy radial shockwave device. However, in other embodiments, different devices may be utilized to apply the electrical shockwave treatment to the treated areas of the patient's heel.

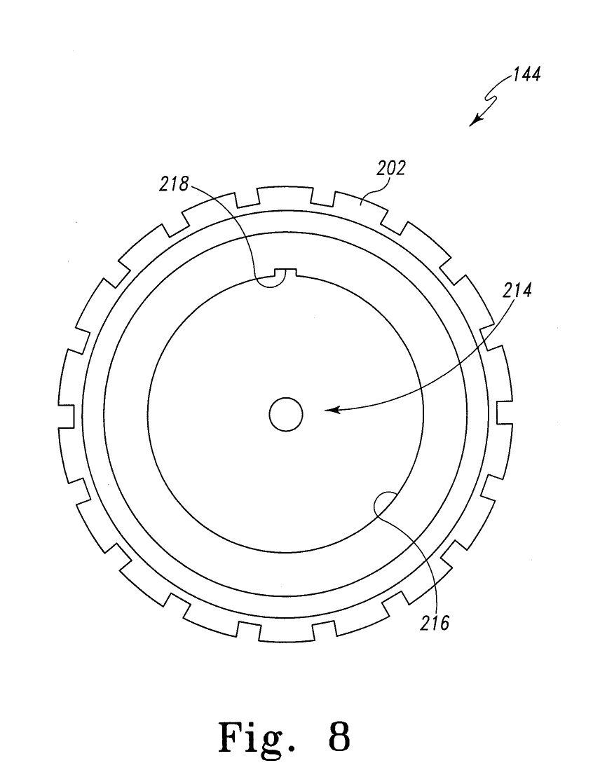

Referring now to FIGS. 6-10, an illustrative tool 200 for performing a partial plantar fasiectomy procedure includes a trocar 202 and a rasp 204. The tool 200 may be used, for example, to excise degenerative areas of the patient's plantar fascia as described above in process step 150 of the algorithm 144 of FIG. 5. As shown in FIGS. 6-8, the trocar 202 includes a cylindrical base 206 and an elongated shaft 208 having a pointed end 210 extending from the base 206. The base 206 is ribbed to improve the grip of the base 206. Additionally, the illustrative trocar 202 includes a thumb rest recess 212 defined in the base 206. The thumb rest recess 212 provides a place for the surgeon's thumb during operation of the tool 200 and also orients the tool 200 when the surgeons thumb is placed thereon. The trocar 202 includes an aperture 214 defined in an end of the base 206. The trocar 202 also includes an internal passageway (not shown) defined in the elongated shaft 208 in communication with the aperture 214 of the base 206. As shown in FIG. 8, the aperture 214 is defined by a cylindrical inner wall 216 of the base 206. An elongated slot 218 is defined in the inner wall 216. The trocar 202 further includes an opening 220 defined in the elongated shaft 208 toward the pointed end 210. The opening 220 is in communication with the internal passageway (not shown) of the elongated shaft 208.

The trocar 202 may be of any size useful for performing a partial plantar fasiectomy on a particular patient. For example, in one particular embodiment, the trocar 202 has an overall length 222 of about 70 millimeters. In such embodiments, the base 206 of the trocar 202 may have a length 224 of about 19.5 millimeters and the elongated shaft 208 may have a length 226 of about 50.5 millimeters. Additionally, in such embodiments, the opening 220 defined in the elongated shaft 208 may have a length 228 of about 6 millimeters and a height 230 of about 1 millimeter.

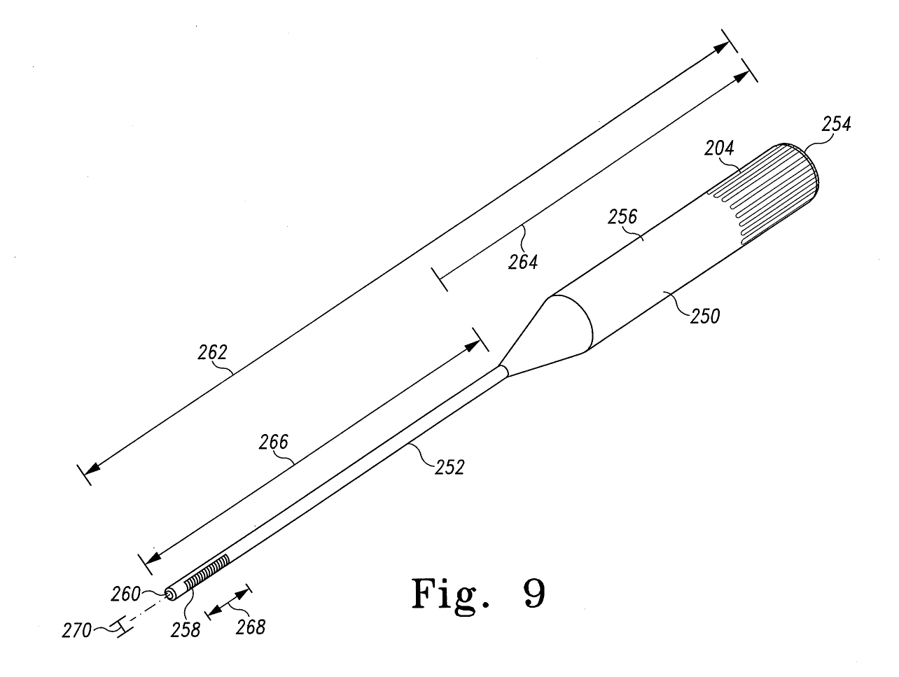

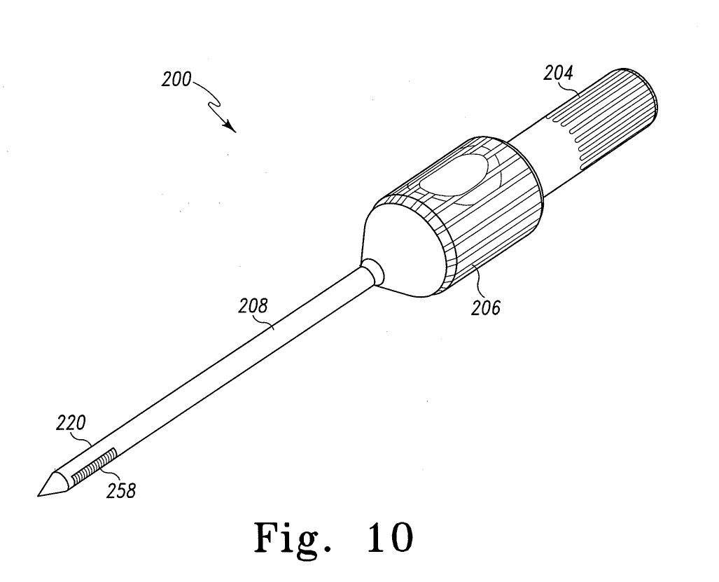

Referring now to FIG. 9, the rasp 204 includes a base 250 and an elongated shaft 252. The base 250 includes a ribbed end 254 and an elongated rail 256 extending upwardly therefrom and extending longitudinally with respect to the base 250. The rasp 204 also includes a file 258 defined toward an end 260 of the elongated shaft 252. As illustrated in FIG. 10, the rasp 204 is configured to be inserted into the trocar 202 during use. As such, the rasp 204 is sized to be received in the internal passageway and aperture 214 of the trocar 202. For example, in one particular embodiment, the rasp 204 has an overall length 262 of about 87.2 millimeters. In such embodiments, the base 250 of the rasp 204 may have a length 264 of about 40.7 millimeters and the elongated shaft 252 may have a length 266 of about 46.5 millimeters. Additionally, in such embodiments, the file 258 is configured to be received in the opening 220 of the elongated shaft 208 of the trocar 202 when the rasp 204 is coupled to the trocar 202. As such, in one particular embodiment, the file 258 may have a length 268 of about 5.84 millimeters and a height 270 of less than about 1 millimeter.

In use, as discussed above, the rasp 204 is configured to be coupled to the trocar 202 as illustrated in FIG. 10. To do so, the elongated shaft 252 of the rasp 204 is inserted into the internal passageway (not shown) defined in the elongated shaft 208 of the trocar 202 via the aperture 214. The rasp 204 is so inserted such that the base 250 of the rasp 204 is received in the aperture 214 of the base 206 of the trocar 202. To do so, the rail 256 defined on the base 250 is received in the elongated slot 218 defined in the inner wall 216 of the base 206 of the trocar 202. As such, the rasp 204 is positioned in a predetermined orientation relative to the trocar 202 via the cooperation of the elongated slot 218 and the rail 256. In such an orientation, the file 268 of the rasp 204 extends through the opening 220 of the trocar 202 as shown in FIG. 10. As such, the rasp 204 may be used to excise degenerative areas of the patient's plantar fascia. In addition, when the rasp 204 is removed from the trocar 202, the trocar 202 may be used to access the patient's plantar fascia during other procedures. For example, the trocar 202 may be used during the injection of growth factors in the process step 152 of the algorithm 144 illustrated in and described above in regard to FIG. 5.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the method and device described herein. It will be noted that alternative embodiments of the method and device of the present disclosure may not include all of the features described yet still benefit from at least some of the advantages of such features. Those of ordinary skill in the art may readily devise their own implementations of the method and device that incorporate one or more of the features of the present invention and fall within the spirit and scope of the present disclosure as defined by the appended claims.

1. A method for treating heel pain of a patient, the method comprising:
determining the number of etiologies of the heel pain of the patient;
determining the time span of the heel pain of the patient;
determining the stage of the heel pain of the patient; and
performing a medical procedure on the patient, the medical procedure being selected based on the determined number of etiologies, the stage, and the severity of the heel pain of the patient, wherein the medical procedure comprises a partial plantar fasciectomy procedure.

2. The method of claim 1, wherein determining the number of etiologies of the heel pain comprises analyzing the patient's responses to a questionnaire designed to identify multiple etiology heel pain syndrome.

3. The method of claim 1, wherein determining the stage of the heel pain comprises determining the time span of the heel pain of the patient.

4. The method of claim 3, wherein determining the stage of the heel pain further comprises determining whether the patient previously received treatment for the heel pain.

5. The method of claim 1, wherein determining the severity of the heel pain comprises:
performing an ultrasound procedure on a heel of the patient, and
determining the thickness of hypoechoic tissue in the heel of the patient using the ultrasound procedure.

6. The method of claim 1, wherein performing the medical procedure comprises:
collecting a sample of the patient's blood and
processing the sample to retrieve a plurality of growth factors.

7. The method of claim 6, wherein performing the medical procedure comprises:
performing an ultrasound procedure on the heel of the patient and
identifying degenerative areas of the plantar fascia of the patient.

8. The method of claim 7, wherein performing the medical procedure comprises excising at least a portion of the degenerative tissue of the patient's plantar fascia with a rasp.

9. The method of claim 8, wherein performing the medical procedure comprises injecting the plurality of growth factors into the patient's plantar fascia.

10. The method of claim 9, wherein performing the medical procedure comprises applying an electrical acoustic shockwave treatment to the patient's plantar fascia.

11. The method of claim 10, wherein performing the medical procedure comprises applying about 2,000 extracorporeal electrical shocks to the patient's plantar fascia.

12. A method for performing a partial plantar fasiectomy procedure, the method comprising:
collecting a sample of a patient's blood;
processing the sample to retrieve growth factors therefrom;
identifying the degenerative areas of the patient's plantar fascia;
excising at least a portion of the degenerative tissue of the patient's plantar fascia;
injecting the patient's growth factors into the patient's plantar fascia; and
applying an electrical shockwave treatment to the patient's plantar fascia.

13. The method of claim 12, wherein identifying the degenerative areas of the patient's plantar fascia comprises performing an ultrasound procedure on a heel of the patient.

14. The method of claim 12, wherein excising the degenerative tissue of the patient's plantar fascia comprises excising the degenerative tissue using a rasp.

15. The method of claim 12, wherein excising the degenerative tissue of the patient's plantar fascia comprises excising the degenerative tissue using a small gauge percutaneous cannulated technique.

16. The method of claim 12, wherein applying an electrical shockwave treatment comprises applying about 2,000 extracorporeal electrical shocks to the patient's plantar fascia.

17. The method of claim 12, wherein injecting the patient's growth factors into the patient's plantar fascia is performed subsequent to excising the degenerative tissue of the patient's plantar fascia.

18. The method of claim 12, wherein applying an electrical acoustic shockwave treatment to the patient's plantar fascia is performed subsequent to identifying the degenerative areas of the patient's plantar fascia, excising the degenerative tissue of the patient's plantar fascia, and injecting the patient's growth factors into the patient's plantar fascia.

19. A surgical tool for use in the performance of a partial planar fasiectomy procedure, the surgical tool comprising:
a trocar having a base and an elongated shaft extending from the base, the elongated shaft having an internal passageway defined therein and an opening defined toward a distal end, the opening being in communication with the internal passageway; and
a rasp coupled to the trocar, the rasp including an elongated shaft and a file defined toward a distal end of the elongated shaft, the elongated shaft being received in the internal passageway of the trocar and the file extending through the opening of the elongated shaft.

20. The surgical tool of claim 19, wherein:
(i) the trocar includes an inner sidewall defining an aperture in the base, the inner sidewall including an elongated slot defined therein, and
(ii) the rasp includes a rail received in the elongated slot of the trocar.
